(19) United States
(12) Patent Application Publication
(10) Pub. No.: US 2005/0063933 A1
(43) Pub. Date: Mar. 24, 2005

(54) Cosmetic hair composition comprising isoeicosane and at least one nonsilicone fixing polymer

(76) Inventors: Sabine Vrignaud, la Plaine St. Denis (FR); Emmanuelle Belli, Asnieres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/868,010

(22) Filed: Jun. 16, 2004

Related U.S. Application Data
(60) Provisional application No. 60/505,162, filed on Sep. 24, 2003.

(30) Foreign Application Priority Data
Jun. 17, 2003 (FR) 03 07288

(51) Int. Cl. A61K 7/06; A61K 7/11

(52) U.S. Cl. 424/70.16

(57) ABSTRACT

The invention relates to a cosmetic hair composition comprising, in a cosmetically acceptable medium, isoeicosane and at least one fixing polymer. It is also directed towards a cosmetic process, in particular a process for fixing and/or holding the hairstyle using said composition, and also the use of this composition for giving the hair a sheen.

This application claims benefit of U.S. Provisional Application No. 60/505,162, filed Sep. 24, 2003.

The present disclosure relates to novel cosmetic hair compositions comprising isoeicosane and at least one nonsilicone fixing polymer. It also relates to a cosmetic process, for instance a process for fixing and/or holding a hairstyle using the compositions disclosed herein, and to the use of these compositions for producing a cosmetic formulation intended, for example, for holding and/or shaping a hairstyle.

It is known practice to use isoeicosane, a nonvolatile hydrocarbon-based oil, as a conditioning agent in cosmetic hair compositions. For example, patent application FR 2,815,853 describes cosmetic hair compositions in the form of a mousse and comprising at least one aminoplast backbone polymer, with isoeicosane as possible conditioning agent.

However, there is a need for hair products that make it possible to give the hair some hold and a long-lasting appearance of sheen, without making it too markedly greasy in nature. Such properties are not always provided by the compositions of the state of the art.

The present disclosure proposes to provide a solution to this need. For instance, the present inventors have found that compositions comprising isoeicosane and at least one nonsilicone fixing polymer can give the hair an intense and persistent sheen and can make the hair particularly smooth to the touch.

It is also disclosed herein that these compositions can be formulated in an alcoholic medium.

Other aspects of the present disclosure will emerge on reading the description and examples which follow.

Disclosed herein, therefore, is a cosmetic hair composition, comprising, in a cosmetically acceptable medium, isoeicosane and at least one nonsilicone fixing polymer.

Also disclosed herein is a cosmetic hair process, for instance, a process for fixing and/or holding a hairstyle using the compositions according to the present disclosure.

Yet another aspect of the present disclosure is the use of the compositions according to the present disclosure, for giving a sheen to the hair.

Isoeicosane is a hydrocarbon that is liquid at ambient temperature. For example, the commercial product sold under the name PERMETHYL 102 A by the company Bayer may be used.

The isoeicosane may be present in the composition in an amount ranging from 0.05 to 20% by weight, relative to the total weight of the composition, for instance from 1 to 10% by weight of the total weight of the composition.

For the purpose of the present disclosure, the term “fixing polymer” means any polymer that makes it possible to give shape to the hair and/or to maintain the shape.

The nonsilicone fixing polymers that may be suitable for use herein include, for example, those chosen from cationic, anionic, amphoteric and nonionic nonsilicone fixing polymers, and mixtures thereof.

The cationic nonsilicone fixing polymers that can be used according to the present disclosure may be chosen from polymers comprising primary, secondary, tertiary and/or quaternary amine groups that are part of the polymer chain or directly connected thereto, and which have a number-average molecular mass ranging from 500 to 5,000,000, such as from 1,000 to 3,000,000.

Among these polymers, non-limiting mention may be made of, for example, the following cationic polymers:
(1) homopolymers or copolymers of acrylic or methacrylic esters or amides, containing amino functions, comprising at least one of the units of formulae below: 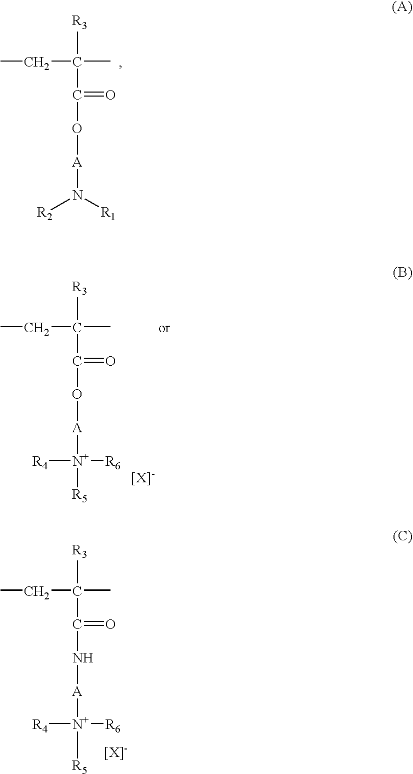
   wherein:
R₁ and R₂, which may be identical or different, are chosen from hydrogen atoms and alkyl groups having from 1 to 6 carbon atoms;
R₃ is chosen from a hydrogen atom and a CH₃ group;
A is chosen from linear and branched alkyl groups comprising from 1 to 6 carbon atoms and hydroxyalkyl groups comprising from 1 to 4 carbon atoms;
R₄, R₅ and R₆, which may be identical or different, are chosen from alkyl groups having from 1 to 18 carbon atoms and benzyl groups;
X is chosen from methosulphate anions and halides such as chloride and bromide.

The copolymers of family (1) also comprise at least one unit derived from comonomers that can be chosen from acrylamides; methacrylamides; diacetone acrylamides; acrylamides; methacrylamides substituted on the nitrogen with lower (C₁₋₄) alkyl groups; groups derived from acrylic or methacrylic acids or esters thereof; vinyllactams such as vinylpyrrolidone or vinylcaprolactam; and vinyl esters.

Thus, among the copolymers of family (1), non-limiting mention may be made of:
copolymers of acrylamide and of dimethylaminoethyl methacrylate, quaternized with dimethyl sulphate or with a dimethyl halide, such as the product sold under the name Hercofloc® by the company Hercules,
copolymers of acrylamide and of methacryloyloxyethyltrimethylammonium chloride described, for example, in patent application EP-A-080976 and sold under the name BINA QUAT P 100 by the company Ciba Geigy,
copolymers of acrylamide and of methacryloyloxyethyltrimethylammonium methosulphate, such as the product sold under the name RETEN by the company Hercules,
quaternized or nonquaternized vinylpyrrolidone/dialkylaminoalkyl acrylate or methacrylate copolymers, such as the products sold under the names “Gafquat®” by the company ISP, such as, for example, “Gafquat® 734” or “Gafquat® 755,” or alternatively the products called “Copolymer® 845, 958 and 937;” these polymers are described in detail in French Patent Nos. 2,077,143 and 2,393,573,
polymers comprising a fatty chain and a vinylpyrrolidone unit, such as the product sold under the name STYLEZE W20 and STYLEZE W10 by the company ISP,
dimethylaminoethyl methacrylate/vinylcaprolactam/vinylpyrrolidone terpolymers, such as the product sold under the name Gaffix® VC 713 by the company ISP, and
quaternized vinylpyrrolidone/dimethylaminopropyl methacrylamide copolymers such as, for instance, the product sold under the name “Gafquat® HS 100” by the company ISP;
(2) cationic polysaccharides, for instance containing quaternary ammonium, such as those described in U.S. Pat. Nos. 3,589,578 and 4,031,307, for instance guar gums comprising trialkylammonium cationic groups. Such products are for example, sold under the trade names JAGUAR C13 S, JAGUAR C 15 and JAGUAR C 17 by the company Meyhall;
(3) quaternized copolymers of vinylpyrrolidone and vinylimidazole;
(4) chitosans or their salts; the salts that may be used include, for example, the acetate, lactate, glutamate, gluconate and pyrrolidonecarboxylate of chitosan. Among these compounds, non-limiting mention may be made of the chitosan having a degree of deacetylation of 90.5% by weight, sold under the name KYTAN BRUT STANDARD by the company Aber Technologies, and the chitosan pyrrolidonecarboxylate sold under the name Kytamer® PC by the company Amerchol;
(5) cationic cellulose derivatives such as the copolymers of cellulose or of cellulose derivatives grafted with a water-soluble quaternary ammonium monomer, and described, for example, in U.S. Pat. No. 4,131,576, such as hydroxyalkylcelluloses, for instance hydroxymethyl-, hydroxyethyl- and hydroxypropyl celluloses grafted for instance, with a methacryloyloxyethyltrimethylammonium, methacrylamidopropyltrimethylammonium or dimethyldiallylammonium salt. The marketed products corresponding to this definition include, as a non-limiting example, the products sold under the names “CELQUAT L 200” and “CELQUAT H 100” by the company National Starch.

The anionic nonsilicone fixing polymers that may be generally used are polymers comprising groups derived from carboxylic, sulphonic and phosphoric acid, and have a number-average molecular mass ranging from 500 to 5,000,000.

The carboxylic groups are provided by unsaturated monocarboxylic or dicarboxylic acid monomers such as those of formula (I): 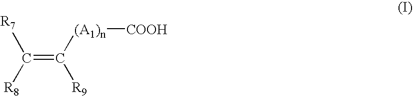
wherein n is an integer ranging from 0 to 10; A₁ is a methylene group optionally linked to the carbon atom of the unsaturated group, or to the neighbouring methylene group when n is greater than 1, by a hetero atom such as oxygen or sulphur; R₇ is chosen from a hydrogen atom and phenyl and benzyl groups; R₈ is chosen from a hydrogen atom, lower alkyl groups and carboxyl groups; R₉ is chosen from a hydrogen atom, lower alkyl groups, —CH₂—COOH groups, phenyl groups and benzyl groups.

In formula (I), a lower alkyl group means a group having from 1 to 4 carbon atoms, such as the methyl and ethyl groups.

Non-limiting examples of anionic nonsilicone fixing polymers comprising carboxylic groups that may be used according to the present disclosure include:
A) homopolymers or copolymers of acrylic or methacrylic acid or their salts, such as the products sold under the names Versicol® E or K by the company Allied Colloid, and Ultrahold® by the company BASF; copolymers of acrylic acid and of acrylamide sold in the form of their sodium salts, under the names RETEN 421, 423 or 425 by the company Hercules; sodium salts of polyhydroxycarboxylic acids.
B) Copolymers of acrylic or methacrylic acid with a monoethylenic monomer such as ethylene, styrene, vinyl esters, esters of acrylic or methacrylic acid, optionally grafted with a polyalkylene glycol such as polyethylene glycol, and optionally crosslinked. Such polymers are described for example in French Patent No.1,222,944 and German application No. 2,330,956; the copolymers of this type containing in their chain an optionally N-alkylated and/or hydroxyalkylated acrylamide unit, as described in particular in Luxembourg patent application Nos. 75370 and 75371, or proposed under the name QUADRAMER by the company American Cyanamid. Non-limiting mention may also be made of copolymers of acrylic acid and of C₁-C₄ alkyl methacrylate and terpolymers of vinylpyrrolidone, of acrylic acid and of C₁-C₂₀ alkyl, for example lauryl, methacrylate, such as the product sold by the company ISP under the name Acrylidone® LM and methacrylic acid/ethyl acrylate/tert-butyl acrylate terpolymers such as the product sold under the name Luvimer® 100 P by the company BASF.

Non-limiting mention may also be made of the methacrylic acid/acrylic acid/ethyl acrylate/methyl methacrylate copolymers in aqueous dispersion, sold under the name Amerhold® DR 25 by the company Amerchol.

C) Crotonic acid copolymers, such as those comprising in their chain vinyl acetate or propionate units, and optionally other monomers such as allyl and methallyl esters, vinyl ether and vinyl esters of a linear or branched, saturated carboxylic acid comprising a long hydrocarbon-based chain, for instance those comprising at least 5 carbon atoms, it being possible for these polymers to be optionally grafted or crosslinked, or another monomer of a vinyl, allyl or methallyl ester of an α- or β-cyclic carboxylic acid. Such polymers are described, inter alia, in French Patent Nos. 1,222,944,1,580,545, 2,265,782, 2,265,781,1,564,110 and 2,439,798. Commercial products that fall into this class are the resins 28-29-30, 26-13-14 and 28-13-10 sold by the company National Starch.

D) Copolymers of monounsaturated C₄-C₈ carboxylic acids or anhydrides, chosen from:
copolymers comprising (i) at least one entity chosen from maleic, fumaric and itaconic acids and anhydrides and (ii) at least one monomer chosen from vinyl esters, vinyl ethers, vinyl halides, phenylvinyl derivatives, and acrylic acid and its esters, wherein the anhydride functions of these copolymers are optionally monoesterified or monoamidated. Such polymers are described for instance in U.S. Pat. Nos. 2,047,398, 2,723,248 and 2,102,113, and GB Patent No. 839,805. Commercial products include, for example, those sold under the names Gantrez® AN or ES by the company ISP;
copolymers comprising (i) at least one unit chosen from maleic, citraconic and itaconic anhydride units and (ii) at least one monomer chosen from allyl and methallyl esters optionally comprising at least one group chosen from acrylamide, methacrylamide, α-olefin, acrylic ester, methacrylic ester, acrylic acid, methacrylic acid, and vinylpyrrolidone groups in their chain,
   the anhydride functional groups of these polymers being optionally monoesterified or monoamidated. Such polymers are, for example, described in French Patent Nos. 2,350,384 and 2,357,241

E) Polyacrylamides comprising carboxylate groups.

The homopolymers and copolymers comprising sulphonic groups are polymers comprising at least one unit chosen from vinylsulphonic, styrenesulphonic, naphthalenesulphonic and acrylamidoalkylsulphonic units.

These polymers can be, for instance, chosen from:
the salts of polyvinylsulphonic acid having a molecular mass ranging from 1,000 to 100,000, and also the copolymers with an unsaturated comonomer such as acrylic or methacrylic acids and their esters, and acrylamide or its derivatives, vinylethers and vinylpyrrolidone;
the salts of polystyrenesulphonic acid such as the sodium salts sold, for example, under the names Flexan® 500 and Flexan® 130 by National Starch. These compounds are described in the French Patent No. 2,198,719;
the salts of polyacrylamidesulphonic acids such as those mentioned in U.S. Pat. No. 4,128,631, for instance, the polyacrylamidoethylpropanesulphonic acid sold under the name COSMEDIA POLYMER HSP 1180 by Henkel.

As another anionic nonsilicone fixing polymer which may be used according to the present disclosure, non-limiting mention may be made of the anionic branched block polymer sold under the name FIXATE G100 by the company Noveon.

According to the present disclosure, the anionic nonsilicone fixing polymers may be, for example, chosen from acrylic acid copolymers such as the acrylic acid/ethyl acrylate/N-tert-butylacrylamide terpolymers sold for instance under the name Ultrahold® Strong by the company BASF; copolymers derived from crotonic acid such as the vinyl acetate/vinyl tert-butylbenzoate/crotonic acid terpolymers and the crotonic acid/vinyl acetate/vinyl neododecanoate terpolymers sold, for example, under the name RÉSINE 28-29-30 by the company National Starch; polymers derived from maleic, fumaric or itaconic acids or anhydrides with vinyl esters, vinyl ethers, vinyl halides, phenylvinyl derivatives, acrylic acid and its esters, such as the methyl vinyl ether/monoesterified maleic anhydride copolymers sold, for example, under the name Gantrez® by the company ISP; the copolymers of methacrylic acid and of methyl methacrylate sold under the name Eudragit® L by the company Rohm Pharma; the copolymers of methacrylic acid and of ethyl acrylate sold under the name Luvimer® MAEX or MAE by the company BASF, and the vinyl acetate/crotonic acid copolymers sold for instance under the name LUVISET CA 66 by the company BASF, and the vinyl acetate/crotonic acid copolymers grafted with polyethylene glycol sold under the name Aristoflex® A by the company BASF, and the polymer sold under the name FIXATE G100 by the company Noveon.

Among the anionic nonsilicone fixing polymers mentioned above, non-limiting mention may be made, in the context of the present disclosure, of the monoesterified methyl vinyl ether/maleic anhydride copolymers sold under the name Gantrez® ES 425 by the company ISP, the acrylic acid/ethyl acrylate/N-tert-butylacrylamide terpolymers sold under the name Ultrahold® Strong by the company BASF, the copolymers of methacrylic acid and of methyl methacrylate sold under the name Eudragit® L by the company Rohm Pharma, the vinyl acetate/vinyl tert-butylbenzoate/crotonic acid terpolymers and the crotonic acid/vinylacetate/vinyl neododecanoate terpolymers sold under the name RESINE 28-29-30 by the company National Starch, the copolymers of methacrylic acid and of ethyl acrylate sold under the name Luvimer® MAEX or MAE by the company BASF, the vinylpyrrolidone/acrylic acid/lauryl methacrylate terpolymers sold under the name Acrylidone® LM by the company ISP, and the polymer sold under the name FIXATE G100 by the company Noveon.

The amphoteric nonsilicone fixing polymers that can be used in accordance with the present disclosure can be chosen from polymers containing B and C units distributed randomly in the polymer chain, wherein B is a unit derived from a monomer comprising at least one basic nitrogen atom and C is a unit derived from an acid monomer comprising at least one group chosen from carboxylic and sulphonic groups, or alternatively, B and C can be chosen from groups derived from zwitterionic monomers of carboxybetaines or of sulphobetaines. B and C can also be chosen from cationic polymer chains comprising primary, secondary, tertiary or quaternary amine groups, in which at least one of the amine groups bears a carboxylic or sulphonic group linked via a hydrocarbon-based group, or alternatively, B and C are part of a chain of a polymer comprising ethylene-α,β-dicarboxylic units, one of the carboxylic groups of which has been brought to react with a polyamine comprising at least one primary or secondary amine group.

Among the amphoteric nonsilicone fixing polymers corresponding to the definition given above that may be used, non-limiting mention may be made of the following polymers:
(1) copolymers containing acidic vinyl units and basic vinyl units, such as those resulting from the copolymerization of a monomer derived from a vinyl compound bearing a carboxylic group such as, for instance, acrylic acid, methacrylic acid, maleic acid or alpha-chloroacrylic acid, and of a basic monomer derived from a substituted vinyl compound comprising at least one basic atom, such as, for instance, dialkylaminoalkyl methacrylate and acrylate, dialkylaminoalkylmethacrylamide and -acrylamide. Such compounds are described in U.S. Pat. No. 3,836,537;
(2) polymers comprising units derived:
   a) from at least one monomer chosen from acrylamides and methacrylamides substituted on the nitrogen atom with an alkyl group,
   b) from at least one acidic comonomer comprising at least one reactive carboxylic group, and
   c) from at least one basic comonomer such as acrylic or methacrylic acid esters containing primary, secondary, tertiary and quaternary amine substituents, and the product of quaternization of dimethylaminoethyl methacrylate with dimethyl or diethyl sulphate.

For example, the N-substituted acrylamides and methacrylamides wherein the compounds in which the alkyl groups contain from 2 to 12 carbon atoms may be used according to the present disclosure, such as N-ethylacrylamide, N-tert-butylacrylamide, N-tert-octylacrylamide, N-octylacrylamide, N-decylacrylamide and N-dodecylacrylamide, and also the corrresponding methacrylamides.

The acidic comonomers may be chosen from, for example, acrylic acid, methacrylic acid, crotonic acid, itaconic acid, maleic acid and fumaric acid, and also the alkyl monoesters, having from 1 to 4 carbon atoms, of maleic or fumaric acids or anhydrides. For further example, the basic comonomers may be aminoethyl, butylaminoethyl, N,N′-dimethylaminoethyl and N-tert-butylaminoethyl methacrylates. In one embodiment of the present disclosure, the copolymers whose CTFA (4th edition, 1991) name is octylacrylamide/acrylates/butylaminoethyl methacrylate copolymer, such as the products sold under the name Amphomer® or Lovocryl® 47 by the company National Starch, can be used;
(3) crosslinked and acylated polyamino amides partially or totally derived from polyamino amides of formula (II): CO—R₁₀—CO— (II)
   wherein R₁₀ is chosen from divalent groups derived from a saturated dicarboxylic acid; mono- and dicarboxylic aliphatic acids containing an ethylenic double bond; esters of a lower alkanol, having 1 to 6 carbon atoms, of these acids; and groups derived from the addition of any one of said acids to a bis(primary) or bis(secondary)amine; and Z is chosen from groups derived from bis(primary), mono- or bis(secondary) polyalkylene-polyamines, for example:
a) present in an amount ranging from 60 to 100 mol %, the group: —NH₄(CH₂)ₓ—NHₚ (III)
   wherein x=2 and p=2 or 3, or alternatively x=3 and p=2, this group being derived from an amine chosen from diethylenetriamine, triethylenetetraamine and dipropylenetriamine;
b) present in an amount ranging from 0 to 40 mol %, the group (III) wherein x=2 and p=1 and which is derived from ethylenediamine, or the group derived from piperazine: 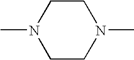
c) present in an amount ranging from 0 to 20 mol %, the group —NH—(CH₂)₆—NH—derived from hexamethylenediamine,
   these polyamino amides being crosslinked by reaction addition of a difunctional crosslinking agent chosen from epihalohydrins, diepoxides, dianhydrides and bis-unsaturated derivatives, using from 0.025 to 0.35 mol of crosslinking agent per amine group of the polyamino amide, and acylated by the action of acrylic acid, chloroacetic acid or an alkane sultone or salts thereof.

The saturated carboxylic acids are may be chosen from for example, acids having from 6 to 10 carbon atoms, such as adipic acid, 2,2,4-trimethyladipic acid and 2,4,4-trimethyladipic acid, terephthalic acid, acids containing an ethylenic double bond, for instance acrylic acid, methacrylic acid and itaconic acid, for example. The alkane sultones used in the acylation can be chosen from, for example, propane sultone and butane sultone, the salts of the acylating agents may be, for instance, the sodium or potassium salts;
(4) polymers containing zwitterionic units of formula (IV): 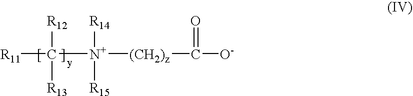
   wherein R₁₁ is a polymerizable unsaturated group such as an acrylate, methacrylate, acrylamide or methacrylamide group, y and z are integers ranging from 1 to 3, R₁₂ and R₁₃ are chosen from hydrogen atoms, methyl, ethyl and propyl groups, R₁₄ and R₁₅ are chosen from hydrogen atoms and alkyl groups such that the sum of the carbon atoms in R₁₄ and R₁₅ does not exceed 10. The polymers comprising such units can also comprise units derived from nonzwitterionic monomers such as dimethyl- or diethylaminoethyl acrylate, methacrylates, alkyl acrylates, alkyl methacrylates, acrylamides, methacrylamides, and vinyl acetate.

By way of non-limiting example, mention may be made of methyl methacrylate/methyl dimethylcarboxymethylammonioethylmethacrylate copolymers, such as the product sold under the name DIAFORMER Z301 by the company Sandoz;
(5) polymers derived from chitosan comprising monomer units corresponding to formulae (D), (E), and (F): 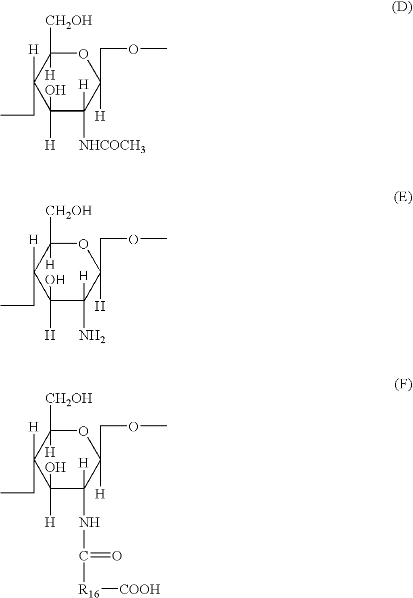
   wherein the unit (D) is present in an amount ranging from 0 to 30%, the unit (E) is present in an amount ranging from 5 to 50%, and the unit (F) is present in an amount ranging from 30 to 90%, it being understood that, in this unit (F), R₁₆ represents a group of formula: 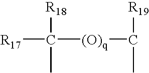
   wherein, if q is equal to zero, R₁₇, R₁₈ and R₁₉, which may be identical or different, are chosen from hydrogen atoms, methyl, hydroxyl, acetoxy, amino, monoalkylamine, and dialkylamine groups, optionally interrupted by at least one nitrogen atom and/or optionally substituted with at least one group chosen from amine, hydroxyl, carboxyl, alkylthio and sulphonic groups, and alkylthio groups in which the alkyl group bears an aminogroup with the proviso that in such an instance, at least one of R₁₇, R₁₈ and R₁₉ is a hydrogen atom;
or, if q is equal to 1, then R₁₇, R₁₈ and R₁₉ are each a hydrogen atom, and also the salts formed by these compounds with bases or acids;
(6) polymers corresponding to general formula (V) are, for example, described in French Patent No. 1,400,366: 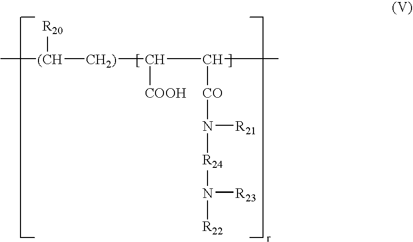
   wherein R₂₀ is chosen from a hydrogen atom, CH₃O groups, CH₃CH₂O groups, and phenyl groups, R₂₁ is chosen from a hydrogen atom and lower alkyl groups such as methyl or ethyl, R₂₂ is chosen from a hydrogen atom and C₁-C₆ lower alkyl groups such as methyl or ethyl, R₂₃ is chosen from C₁-C₆ lower alkyl groups such as methyl or ethyl, and —R₂₄—N(R₂₂)₂ groups, wherein R₂₄ is chosen from —CH₂—CH₂—, —CH₂—CH₂—CH₂— and —CH₂—CH(CH₃)— groups, R₂₂ being as defined above;
(7) polymers derived from the N-carboxyalkylation of chitosan, such as N-carboxymethylchitosan and N-carboxybutylchitosan, sold under the name “EVALSAN” by the company Jan Dekker;
(8) amphoteric polymers of the type —D—X—D—X chosen from:
   a) polymers obtained by the action of chloroacetic acid or sodium chloroacetate on compounds comprising at least one unit of formula (VI):
      —D—X—D—X—D— (VI)
      wherein D is the group: 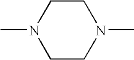
      and X is chosen from E and E′, wherein E and E′, which may be identical or different, are divalent groups chosen from straight and branched chain alkylene groups comprising up to 7 carbon atoms in the main chain, optionally substituted with hydroxyl groups, and which can comprise oxygen, nitrogen and sulphur atoms, and 1 to 3 aromatic and/or heterocyclic rings; wherein the oxygen, nitrogen and sulphur atoms are present in the form of ether, thioether, sulphoxide, sulphone, sulphonium, alkylamine alkenylamine, hydroxyl, benzylamine, amine oxide, quaternary amine, amide, imide, alcohol, ester and/or urethane groups;
   b) polymers of formula:
      —D—X—D—X— (VI′)
      wherein D is the group: 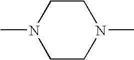
      and X is chosen from E and E′, with the proviso that X is at least once E′; E is as defined above and E′ is a divalent group chosen from straight and branched chain alkylene groups having up to 7 carbon atoms in the main chain, which is optionally substituted with at least one hydroxyl group and comprising at least one nitrogen atom, the nitrogen atom being substituted with an alkyl chain that is optionally interrupted by an oxygen atom and necessarily comprises at least one functional group chosen from carboxyl and hydroxyl functional groups, betainized by reaction with chloroacetic acid or sodium chloroacetate;
(9) (C₁-C₅)alkyl vinyl ether/maleic anhydride copolymers partially modified by semiamidation with an N,N-dialkylaminoalkylamine such as N,N-dimethylamino-propylamine, or by semiesterification with an N,N-dialkylaminoalkanol. These copolymers can also contain other vinyl comonomers such as vinylcaprolactam.

Among the amphoteric nonsilicone fixing polymers described above that may be used according to the present disclosure, non-limiting mention may be made of those of family (3), such as copolymers whose CTFA name is octylacrylamide/acrylates/butylaminoethyl methacrylate copolymer, such as the products sold under the names Amphomer®, Amphomer® LV 71 or Lovocryl®47 by the company National Starch and those of family (4), such as methyl methacrylate/methyl dimethylcarboxymethylammonioethylmethacrylate copolymers sold, for example, under the name Diaformer® Z301 by the company Sandoz.

Further non-limiting examples of nonionic nonsilicone fixing polymers that can be used according to the present disclosure may be chosen, for example, from:
polyalkyloxazolines;
vinyl acetate homopolymers;
vinyl acetate copolymers such as, for example, copolymers of vinyl acetate and of acrylic ester, copolymers of vinyl acetate and of ethylene, and copolymers of vinyl acetate and of maleic ester, for example of dibutyl maleate;
homopolymers and copolymers of acrylic esters, for instance copolymers of alkyl acrylates and of alkyl methacrylates, such as the products proposed by the company Rohm & Haas under the names Primal® AC-261 K and Eudragit® NE 30 D, by the company BASF under the name 8845, or by the company Hoechst under the name Appretan® N9212;
copolymers of acrylonitrile and of a nonionic monomer chosen, for example, from butadiene and alkyl(meth)acrylates; mention may be made of the products provided under the name CJ 0601 B by the company Rohm & Haas;
styrene homopolymers;
styrene copolymers, for instance copolymers of styrene and of alkyl (meth)acrylate, such as the products Mowilith® LDM 6911, Mowilith® DM 611 and Mowilith® LDM 6070 provided by the company Hoechst, the products Rhodopas® SD 215 and Rhodopas® DS 910 provided by the company Rhone Poulenc; copolymers of styrene, of alkyl methacrylate and of alkyl acrylate; copolymers of styrene and of butadiene; or copolymers of styrene, of butadiene and of vinylpyridine;
polyamides;
vinyllactam homopolymers different from the vinylpyrrolidone homopolymers, such as the polyvinylcaprolactam sold under the name Luviskol® Plus by the company BASF; and
vinyllactam copolymers such as a poly(vinylpyrrolidone/vinyllactam) copolymer sold under the trade name Luvitec® VPC 55K65W by the company BASF, poly-(vinylpyrrolidone/vinyl acetate) copolymers such as those sold under the name PVPVA® S630L by the company ISP, and Luviskol® VA 73, VA 64, VA 55, VA 37 and VA 28 by the company BASF; and poly(vinylpyrrolidone/vinyl acetate/vinyl propionate) terpolymers such as, for example, the product sold under the name Luviskol® VAP 343 by the company BASF.

The alkyl groups in the nonionic polymers mentioned above may have for example, from 1 to 6 carbon atoms.

Nonsilicone fixing polymers that may also be used include functionalized or nonfunctionalized, cationic, nonionic, anionic or amphoteric polyurethanes, and mixtures thereof.

The polyurethanes that may be used in the present disclosure also include those described in patent application Nos. EP 0 751,162, EP 0 637,600, EP 0 648,485 and FR 2,743,297, and also in patent application Nos. EP 0 656,021, WO 94/03510, and EP 0619,111.

Among the polyurethanes that may be used in the present disclosure, non-limiting mention may be made of the product sold under the name Luviset Pur® by the company BASF.

In the compositions of the present disclosure, the at least one nonsilicone fixing polymer may be present in an amount ranging from 0.01 to 20%, for instance, from 0.05 to 15%, such as from 0.1 to 10%, by weight, relative to the total weight of the composition

The cosmetically acceptable medium may comprise, for example, water, or at least one cosmetically acceptable solvent, such as alcohols, or water-solvent(s) mixtures. For instance, these cosmetically acceptable solvents may be C₁-C₄ alcohols.

Among these alcohols, mention may be made of ethanol and isopropanol, ethanol being particularly preferred.

The composition according to the invention can also comprise at least one additive chosen from nonionic, anionic, cationic, amphoteric and zwitterionic surfactants; fragrances; screening agents; preserving agents; proteins; vitamins; nonionic, anionic, cationic, amphoteric and zwitterionic polymers other than the nonsilicone fixing polymers described above; mineral, plant and synthetic oils; and any other additive or agent conventionally used in cosmetic compositions, such as anti-dandruff agents, anti-hairloss agents, dyes, pigments, moisturizers such as glycerol and the like, polyols, and reducing agents.

These additives may be present in the composition in an amount ranging from 0.001 to 20% by weight of the total weight of the composition. The precise amount of each additive depends on its nature and is readily determined by those skilled in the art, and will depend on the chosen hair application. Of course, those skilled in the art will take care to choose any additional additive in such a way that the advantageous properties of the composition in accordance with the present disclosure are not, or are not substantially, altered by the envisaged addition.

The compositions as disclosed herein can be used for producing many hair products, such as, for example, products for fixing and/or holding the hair, conditioning products, and haircare products.

These compositions can be packaged in various forms, for example, in pump-dispenser bottles or in aerosol containers, in order to allow the composition to be applied in sprayed form or in mousse form. Such packaging forms are indicated, for example, when it is desired to obtain a spray, a lacquer, or a mousse for fixing or treating the hair. The compositions in accordance with the present disclosure can also be in the form of creams, gels, emulsions or lotions.

When the composition as disclosed herein is packaged in the form of an aerosol for the purpose of obtaining a lacquer or a mousse, it comprises at least one propellant which can be chosen from volatile hydrocarbons such as n-butane, propane, isobutane, and pentane; and halogenated hydrocarbons. Also as a propellant, use may also be made of carbon dioxide, nitrous oxide, dimethyl ether (DME), nitrogen and compressed air. Mixtures of propellants may also be used. In one embodiment of the present disclosure, dimethyl ether is used.

The propellant may be present in the composition, for example in an amount ranging from 5 to 90% by weight, relative to the total weight of the composition in the aerosol device, such as ranging from 10 to 60%.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The following examples are intended to illustrate the invention in a non-limiting manner.

### EXAMPLE 1

Composition A described below was applied to a lock of natural euro-chestnut hair of 5 grams.

Composition A:

| | |
|---|---|
| Permethyl 102 A (1) | 7% |
| Ultra Hold Strong (2) | 5% |
| Ethanol | qs 100% |

(1) isoeicosane sold by the company Bayer
(2) anionic nonsilicone fixing polymer sold by the company BASF (acrylic acid/ethyl acrylate/N-tert-butylacrylamide terpolymer).

Composition A was applied to natural hair using a pump-dispenser bottle. After drying, it was observed that the lock had a sheen, was soft to the touch, and had a pleasant appearance.

### EXAMPLE 2

Composition B described below was applied to a lock of natural euro-chestnut hair of 5 grams.

Composition B:

| | |
|---|---|
| Permethyl 102 A (1) | 10% |
| Pecosil PS-100 (2) | 0.5% |
| Synthalen K (3) | 0.7% |
| Aristoflex A 60 (4) | 0.5% |
| Triethanolamine | 0.6% |
| Water | qs 100% |

(1) isoeicosane sold by the company Bayer
(2) oxyethylenated polydimethylsiloxane containing phosphate groups, sold by the company Phoenix
(3) polyacrylic acid sold by the company 3 V
(4) vinyl acetate/crotonic acid/PEG copolymer sold by the company Clariant

Composition B was applied to natural hair using a pump-dispenser bottle. After drying, it was observed that the lock had good hold, had a sheen, was soft to the touch, and had a pleasant appearance.

1. A cosmetic hair composition comprising, in a cosmetically acceptable medium, isoeicosane and at least one nonsilicone fixing polymer chosen from anionic, amphoteric, nonionic and cationic nonsilicone fixing polymers, wherein the cationic polymers are chosen from:
homopolymers and copolymers chosen from homopolymers and copolymers of acrylic esters, methacrylic esters, acrylic amides, and methacrylic amides, said homopolymers and copolymers containing at least one amino functional group, comprising at least one unit of formula (A): 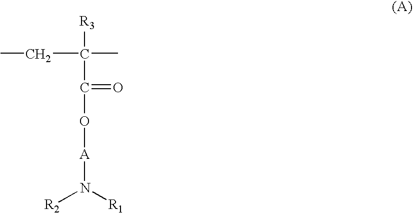
wherein:
R₁ and R₂, which may be identical or different, are chosen from hydrogen atoms and alkyl groups having from 1 to 6 carbon atoms;
R₃ is chosen from a hydrogen atom and CH₃ groups;
A is chosen from linear and branched alkyl groups comprising from 1 to 6 carbon atoms and hydroxyalkyl groups comprising from 1 to 4 carbon atoms;
cationic polysaccharides,
quaternized copolymers of vinylpyrrolidone and vinylimidazole,
chitosans or their salts, and
cationic cellulose derivatives.

2. The composition according to claim 1, wherein the isoeicosane is present in the composition in an amount ranging from 0.05 to 20% by weight, relative to the total weight of the composition.

3. The composition according to claim 2, wherein the isoeicosane is present in the composition in an amount ranging from 1 to 10% by weight, relative to the total weight of the composition.

4. The composition according to claim 1, wherein the at least one anionic nonsilicone fixing polymer is chosen from homopolymers and copolymers of acrylic and methacrylic acid and the salts thereof, crotonic acid copolymers, copolymers of monounsaturated C₄-C₈ carboxylic acids and anhydrides, polyacrylamides comprising carboxylate groups, homopolymers and copolymers comprising sulphonic groups, and anionic polyurethanes.

5. The composition according to claim 1, wherein the at least one amphoteric nonsilicone fixing polymer is chosen from copolymers comprising acidic vinyl units and basic vinyl units, crosslinked and acylated polyamino amides, polymers comprising zwitterionic units, polymers derived from chitosan, modified (C₁-C₅)alkyl vinyl ether/maleic anhydride copolymers, and amphoteric polyurethanes.

6. The composition according to claim 1, wherein the at least one nonionic nonsilicone fixing polymer is chosen from polyalkyloxazolines, vinyl acetate homopolymers and copolymers, homopolymers and copolymers of acrylic esters, acrylonitrile copolymers, styrene homopolymers and copolymers, polyamides, vinyllactam homopolymers different from the vinylpyrrolidone homopolymers, vinyllactam copolymers, and nonionic polyurethanes.

7. The composition according to claim 1, wherein the at least one nonsilicone fixing polymer is present in the composition in an amount ranging from 0.01 to 20% by weight, relative to the total weight of the composition.

8. The composition according to claim 7, wherein the at least one nonsilicone fixing polymer is present in the composition in an amount ranging from 0.05 to 15% by weight, relative to the total weight of the composition.

9. The composition according to claim 8, wherein the at least one nonsilicone fixing polymer is present in the composition in an amount ranging from 0.1 to 10% by weight, relative to the total weight of the composition.

10. The composition according to claim 1, further comprising at least one additive chosen from nonionic, anionic, cationic, amphoteric and zwitterionic surfactants; fragrances; screening agents; preserving agents; proteins; vitamins; nonionic, anionic, cationic and zwitterionic polymers other than the nonsilicone fixing polymers different from those that may already be present; mineral, plant and synthetic oils; anti-dandruff agents; anti-hairloss agents; dyes; pigments; moisturizers; and reducing agents.

11. A cosmetic process for holding a hairstyle, comprising applying to hair a composition comprising, in a cosmetically acceptable medium and in a combined amount effective to hold a hairstyle, isoeicosane and at least one nonsilicone fixing polymer chosen from anionic, amphoteric, nonionic and cationic nonsilicone fixing polymers, wherein the cationic polymers are chosen from:
homopolymers and copolymers chosen from homopolymers and copolymers of acrylic esters, methacrylic esters, acrylic amides, and methacrylic amides, said homopolymers and copolymers containing at least one amino functional group, comprising at least one unit of formula(A): 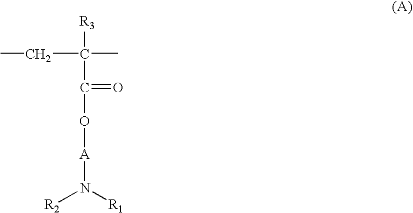
wherein:
R₁ and R₂, which may be identical or different, are chosen from hydrogen atoms and alkyl groups having from 1 to 6 carbon atoms;
R₃ is chosen from a hydrogen atom and CH₃ groups;
A is chosen from linear and branched alkyl groups comprising from 1 to 6 carbon atoms and hydroxyalkyl groups comprising from 1 to 4 carbon atoms;
cationic polysaccharides;
quaternized copolymers of vinylpyrrolidone and vinylimidazole;
chitosans or their salts; and
cationic cellulose derivatives.

12. The process according to claim 11, wherein the isoeicosane is present in the composition in an amount ranging from 0.05 to 20% by weight, relative to the total weight of the composition.

13. The process according to claim 11, wherein the at least one anionic nonsilicone fixing polymer is chosen from homopolymers and copolymers of acrylic and methacrylic acid and the salts thereof, crotonic acid copolymers, copolymers of monounsaturated C₄-C₈ carboxylic acids and anhydrides, polyacrylamides comprising carboxylate groups, homopolymers and copolymers comprising sulphonic groups, and anionic polyurethanes.

14. The process according to claim 11, wherein the at least one amphoteric nonsilicone fixing polymer is chosen from copolymers comprising acidic vinyl units and basic vinyl units, crosslinked and acylated polyamino amides, polymers comprising zwitterionic units, polymers derived from chitosan, modified (C₁-C₅)alkyl vinyl ether/maleic anhydride copolymers, and amphoteric polyurethanes.

15. The process according to claim 11, wherein the at least one nonionic nonsilicone fixing polymer is chosen from polyalkyloxazolines, vinyl acetate homopolymers and copolymers, homopolymers and copolymers of acrylic esters, acrylonitrile copolymers, styrene homopolymers and copolymers, polyamides, vinyllactam homopolymers different from the vinylpyrrolidone homopolymers, vinyllactam copolymers, and nonionic polyurethanes.

16. The process according to claim 11, wherein the at least one nonsilicone fixing polymer is present in the composition in an amount ranging from 0.01 to 20% by weight, relative to the total weight of the composition.

17. The process according to claim 11, wherein the composition further comprises at least one additive chosen from nonionic, anionic, cationic, amphoteric and zwitterionic surfactants; fragrances; screening agents; preserving agents; proteins; vitamins; nonionic, anionic, cationic and zwitterionic polymers other than the nonsilicone fixing polymers different from those that may already be present; mineral, plant and synthetic oils; anti-dandruff agents; anti-hairloss agents; dyes; pigments; moisturizers; and reducing agents.

18. A cosmetic process for giving hair a sheen, comprising applying to hair a composition comprising, in a cosmetically acceptable medium and in a combined amount effective for providing a sheen to the hair, isoeicosane and at least one nonsilicone fixing polymer chosen from anionic, amphoteric, nonionic and cationic nonsilicone fixing polymers, wherein the cationic polymers are chosen from:
homopolymers and copolymers chosen from homopolymers and copolymers of acrylic esters, methacrylic esters, acrylic amides, and methacrylic amides, said homopolymers and copolymers containing at least one amino functional group, comprising at least one unit of formula(A): 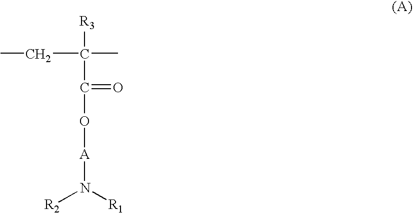
wherein:
R₁ and R₂, which may be identical or different, are chosen from hydrogen atoms and alkyl groups having from 1 to 6 carbon atoms;
R₃ is chosen from a hydrogen atom and CH₃ groups;
A is chosen from linear and branched alkyl groups comprising from 1 to 6 carbon atoms and hydroxyalkyl groups comprising from 1 to 4 carbon atoms;
cationic polysaccharides;
quaternized copolymers of vinylpyrrolidone and vinylimidazole;
chitosans or their salts; and
cationic cellulose derivatives.
